(19) United States
(12) Patent Application Publication
(10) Pub. No.: US 2026/0140108 A1
(43) Pub. Date: May 21, 2026

(54) EXTRACTION ROD AND PREPARATION METHOD THEREOF, AND METHOD FOR DETECTING 25-HYDROXYVITAMIN D

(71) Applicant: Innovation Center of Yangtze River Delta, ZJU, Jiaxing (CN)

(72) Inventors: Fangxing Yang, Jiaxing (CN); Tongqing Deng, Jiaxing (CN)

(21) Appl. No.: 19/052,662

(22) Filed: Feb. 13, 2025

(30) Foreign Application Priority Data
Nov. 15, 2024 (CN) 202411631058.6

(51) Int. Cl. G01N 33/531; G01N 30/02; G01N 30/04; G01N 30/06; G01N 30/30; G01N 30/72

(52) U.S. Cl. CPC G01N 33/531; G01N 30/06; G01N 30/30; G01N 30/72; G01N 2030/027; G01N 2030/045

(57) ABSTRACT

Disclosed are an extraction rod and a preparation method thereof, and a method for detecting 25-hydroxyvitamin D, belonging to the technical field of analysis and detection. The preparation method includes: subjecting a glass rod to etching, silanization, a heat treatment, and activation in sequence to obtain an activated glass rod; placing the activated glass rod in a 25-hydroxyvitamin D antibody solution, and conducting coupling to obtain an antibody-coupled glass rod; and subjecting the antibody-coupled glass rod to deactivation and covalent bond fixation in sequence to obtain the extraction rod.

### CROSS REFERENCE TO RELATED APPLICATION

This patent application claims the benefit and priority of Chinese Patent Application No. 2024116310586, entitled “EXTRACTION ROD AND PREPARATION METHOD THEREOF, AND METHOD FOR DETECTING 25-HYDROXYVITAMIN D” filed on Nov. 15, 2024, the disclosure of which is incorporated by reference herein in its entirety as part of the present application.

### TECHNICAL FIELD

The present disclosure relates to the technical field of analysis and detection, and in particular to an extraction rod and a preparation method thereof, and a method for detecting 25-hydroxyvitamin D.

### BACKGROUND

At present, 25-hydroxyvitamin D is generally detected in clinical practice by liquid chromatography-tandem mass spectrometry (LC-MS/MS), which could distinguish 25-hydroxyvitamin D2 from 25-hydroxyvitamin D3 with excellent sensitivity and accuracy. When the LC-MS/MS is used to detect 25-hydroxyvitamin D, sample pretreatment processes mainly include protein precipitation, liquid-liquid extraction, and solid phase extraction (where common fillers include C18 and polystyrene-divinylbenzene). However, these pretreatment processes may extract many unnecessary non-target substances, thereby causing a strong matrix effect and affecting accuracy of the test results.

### SUMMARY

An object of the present disclosure is to provide an extraction rod and a preparation method thereof, and a method for detecting 25-hydroxyvitamin D. In the present disclosure, the extraction rod is used to detect the 25-hydroxyvitamin D, with small matrix interference, great specificity, and accurate detection results.

To achieve the above object, the present disclosure provides the following technical solutions:

The present disclosure provides a method for preparing an extraction rod, including the following steps:
subjecting a glass rod to etching, silanization, a heat treatment, and activation in sequence to obtain an activated glass rod;
placing the activated glass rod in a 25-hydroxyvitamin D antibody solution, and conducting coupling to obtain an antibody-coupled glass rod; and
subjecting the antibody-coupled glass rod to deactivation and covalent bond fixation in sequence to obtain the extraction rod.

In some embodiments, a 25-hydroxyvitamin D antibody in the 25-hydroxyvitamin D antibody solution has a concentration of 0.1 mg/mL to 0.6 mg/mL; and the 25-hydroxyvitamin D antibody is selected from the group consisting of a 25-hydroxyvitamin D monoclonal antibody and a 25-hydroxyvitamin D polyclonal antibody.

In some embodiments, the coupling is conducted at a temperature of 30° C. to 40° C. for 1 h to 4 h.

The present disclosure further provides an extraction rod prepared by the method as described in above technical solutions.

The present disclosure further provides a method for detecting 25-hydroxyvitamin D in a non-diagnostic purpose, including the following steps:
mixing a biological sample to be tested with a dissociation agent, and subjecting a resulting mixture to dissociation to obtain a dissociation liquid; where the dissociation agent includes 8-anilino-1-naphthalenesulfonic acid and perfluorooctanoic acid (PFOA);
placing the extraction rod as described in above technical solutions in the dissociation liquid, and conducting extraction to obtain an enriched extraction rod;
subjecting the enriched extraction rod to cleaning and elution in sequence to obtain a solution to be tested;
subjecting the solution to be tested to liquid chromatography-tandem mass spectrometry (LC-MS/MS) detection to obtain a chromatogram of the solution to be tested; and
obtaining a content of the 25-hydroxyvitamin D in the biological sample to be tested according to a standard curve of the 25-hydroxyvitamin D and the chromatogram of the solution to be tested.

In some embodiments, the 25-hydroxyvitamin D includes 25-hydroxyvitamin D2 and 25-hydroxyvitamin D3.

In some embodiments, in the dissociation agent, the 8-anilino-1-naphthalenesulfonic acid has a concentration of 0.1 wt % to 0.5 wt % and the PFOA has a concentration of 1 wt % to 5 wt %; the dissociation agent has a pH value of 7.0 to 7.5; and the dissociation is conducted at a temperature of 30° C. to 40° C. for 10 min to 30 min.

In some embodiments, the extraction is conducted at a temperature of 30° C. to 40° C. for 5 min to 30 min;

an eluent for the elution includes one selected from the group consisting of methanol, acetonitrile, and a solution of a derivating agent, and the derivating agent includes one selected from the group consisting of 4-phenyl-1,2,4-triazoline-3,5-dione (PTAD), 4-(2-(6,7-dimethoxy-4-methyl-3-oxo-3,4-dihydro-quinoxalin-2-yl)-ethyl)-(1,2,4) triazoline-3,5-di one (DMEQ-TAD), and 4-(4′-dimethylaminophenyl)-1,2,4-triazoline-3,5-dione (DAPTAD); and the elution is conducted at a temperature of 30° C. to 40° C. for 10 min to 30 min.

In some embodiments, conditions of liquid phase chromatography in the LC-MS/MS detection includes:
a mobile phase including a mobile phase A and a mobile phase B, the mobile phase A being an aqueous ammonium acetate solution with a concentration of 2 mmol/L, and the mobile phase B being a solution of ammonium acetate in methanol with a concentration of 2 mmol/L; and
a gradient elution program including: a volume fraction of the mobile phase B being 10% from 0 min to 1 min; increasing the volume fraction of the mobile phase B from 10% to 100% from 1 min to 3.5 min; maintaining the volume fraction of the mobile phase B at 100% from 3.5 min to 4.5 min; decreasing the volume fraction of the mobile phase B from 100% to 10% from 4.5 min to 4.6 min; and maintaining the volume fraction of the mobile phase B at 10% from 4.6 min to 5 min.

In some embodiments, conditions of mass spectrometry in the LC-MS/MS detection includes:

an acquisition ion mode: electrospray ionization (ESI) positive ion mode; a capillary voltage: 3.6 kV; a desolvation gas temperature: 500° C.; a desolvation gas flow rate: 1,000 L/h; a cone gas flow rate: 150 L/h; and a scanning mode: multiple reaction monitoring mode.

Beneficial effects of some embodiments of the represent disclosure: the method includes the following steps: subjecting a glass rod to etching, silanization, a heat treatment, and activation in sequence to obtain an activated glass rod; placing the activated glass rod in a 25-hydroxyvitamin D antibody solution, and conducting coupling to obtain an antibody-coupled glass rod; and subjecting the antibody-coupled glass rod to deactivation and covalent bond fixation in sequence to obtain the extraction rod. The extraction rod (i.e., vitamin D antibody immunoaffinity extraction rod) is prepared based on the glass rod by antibody immuno-enrichment, and the 25-hydroxyvitamin D is extracted with the extraction rod and then tested. The extraction rod shows small matrix interference, great specificity, and excellent accuracy; and the extraction rod is simple to use and easy to achieve automated operation, thus being particularly suitable for the detection on large batches of clinical samples.

### BRIEF DESCRIPTION OF THE DRAWINGS

FIG. 1 shows a standard curve of the 25-hydroxyvitamin D2.

FIG. 2 shows a standard curve of the 25-hydroxyvitamin D3.

FIG. 3 shows a chromatogram obtained by LC-MS/MS detection of the 25-hydroxyvitamin D2 in a spiked serum sample after extraction with the extraction rod.

FIG. 4 shows a chromatogram obtained by LC-MS/MS detection of the 25-hydroxyvitamin D3 in a spiked serum sample after extraction with the extraction rod.

### DETAILED DESCRIPTION OF THE EMBODIMENTS

The present disclosure provides a method for preparing an extraction rod, including the following steps:

subjecting a glass rod to etching, silanization, a heat treatment, and activation in sequence to obtain an activated glass rod;

placing the activated glass rod in a 25-hydroxyvitamin D antibody solution, and conducting coupling to obtain an antibody-coupled glass rod; and

subjecting the antibody-coupled glass rod to deactivation and covalent bond fixation in sequence to obtain the extraction rod.

In the present disclosure, the extraction rod (i.e., vitamin D antibody immunoaffinity extraction rod) is prepared based on antibody immuno-enrichment, and 25-hydroxyvitamin D is extracted with the extraction rod and then detected. The extraction rod shows small matrix interference, great specificity, and excellent accuracy; and the extraction rod is simple to use and easy to achieve automated operation, thus being particularly suitable for the detection on large batches of clinical samples. The method for preparing the extraction rod according to the present disclosure is described in detail below.

In the present disclosure, unless otherwise specified, the raw materials used are all commercially-available commodities well known to those skilled in the art or prepared by methods well known to those skilled in the art.

In the present disclosure, a glass rod is subjected to etching to obtain an etched glass rod. In an embodiment, the glass rod is a quartz glass rod or a high borosilicate glass rod; the glass rod may have a diameter of 3 mm and a length of 60 mm. Using the glass rod to prepare the extraction rod has lower cost and is easier to obtain than other materials such as stainless steel. As an embodiment, the glass rod is placed in a piranha solution for etching. For example, one end of the glass rod (referred to as a treatment end) is placed in the piranha solution for etching, and an immersion depth of the treatment end in the piranha solution may be not less than 0.5 cm, particularly 1 cm to 1.5 cm. The piranha solution may be prepared from an aqueous hydrogen peroxide solution and concentrated sulfuric acid. The aqueous hydrogen peroxide solution may have a concentration of 30 wt %, and the concentrated sulfuric acid may have a concentration of 86 wt %. A volume ratio of the aqueous hydrogen peroxide solution to the concentrated sulfuric acid may be 3:7. In an embodiment, the etching is conducted at room temperature (25° C.); the etching is conducted for 0.5 h to 1.5 h, specifically 1 h. The etching could remove organic matters on a surface of the glass rod, and active hydroxyl groups could be introduced into the glass rod; after the etching, the etched glass rod is preferably rinsed thoroughly with pure water, anhydrous ethanol, and pure water in sequence.

In the present disclosure, after obtaining the etched glass rod, the etched glass rod is subjected to silanization to obtain a silanized glass rod. In an embodiment, the etched glass rod is placed in a silane modification solution and subjected to silanization. For example, a treatment end of the etched glass rod may be placed in the silane modification solution for silanization, and an immersion depth of the treatment end in the silane modification solution may be not less than 0.5 cm, particularly 1 cm to 1.5 cm; the silane modification solution may be prepared from a silane reagent, water, and ethanol; a volume ratio of the silane reagent, the water, and the ethanol is 5:5: 90. In an embodiment, the silane reagent may be 3-aminopropyltriethoxysilane (APTES), which includes one amino functional group and three ethoxy functional groups in its chemical structure. These functional groups can react with active hydroxyl groups on the surface of the etched glass rod, thereby introducing amino groups on the glass rod. In an embodiment, the silane reagent may also be dihydro-3-[3-(triethoxysilyl)propyl]furan-2,5-dione (TESPSA), which could result in carboxylation of the surface of the glass rod. In an embodiment, the silane reagent may also be 3-(2,3-epoxypropoxy)propyltrimethoxysilane (GPTMS), which could hydroxylate the surface of the glass rod. In an embodiment, the silanization may be conducted at a temperature of 20° C. to 30° C., specifically at room temperature; the silanization may be conducted for 20 h to 30 h, specifically 24 h. In some embodiments, the method further includes after the silanization is completed, rinsing the silanized glass rod thoroughly with pure water and anhydrous ethanol in sequence.

In the present disclosure, the silanized glass rod is subjected to a heat treatment to obtain a heat-treated glass rod. In an embodiment, the heat treatment may be conducted at a temperature of 75° C. to 85° C., specifically 80° C.; the heat treatment may be conducted for 10 h to 15 h, specifically 12 h; the heat treatment is preferably conducted in a protective atmosphere, which may be nitrogen. Taking the APTES being the silane reagent as an example, amino group fixation could be achieved through the heat treatment, namely the amino groups introduced on the glass rod are more stable. In an embodiment, the heat treatment may be conducted in a tubular furnace; the method preferably includes after the heat treatment is completed, taking out the heat-treated glass rod and then cooling.

In the present disclosure, the heat-treated glass rod is subjected to activation to obtain an activated glass rod. In an embodiment, the heat-treated glass rod is placed in an activator for activation, for example, a treatment end of the heat-treated glass rod may be placed in the activator for activation, and an immersion depth of the treatment end in the activator may be not less than 0.5 cm, specifically 1 cm to 1.5 cm. In an embodiment, the activator may be prepared from glutaraldehyde and PBS solution, and the glutaraldehyde in the activator may have a concentration of 2 wt % to 3 wt %, specifically 2.5 wt %; the PBS solution may have a pH value of 7.0 to 7.4, specifically 7.0 in an example. In an embodiment, the activation may be conducted at a temperature of 20° C. to 30° C., specifically at room temperature; the activation may be conducted for 4 h to 6 h, specifically 5 h; the method preferably includes after the activation is completed, rinsing the activated glass rod thoroughly with pure water. In an embodiment, the activator is prepared using the glutaraldehyde as an example, and amino groups on the heat-treated glass rod can react with aldehyde groups on the glutaraldehyde, thereby introducing aldehyde groups on the glass rod.

In the present disclosure, after obtaining the activated glass rod, the activated glass rod is placed in a 25-hydroxyvitamin D antibody solution, and coupling is conducted to obtain an antibody-coupled glass rod. In an embodiment, a treatment end of the activated glass rod may be placed in the 25-hydroxyvitamin D antibody solution for coupling, and an immersion depth of the treatment end in the 25-hydroxyvitamin D antibody solution may be 0.5 cm to 1 cm, specifically not exceeding the immersion depth in other steps. In an embodiment, a 25-hydroxyvitamin D antibody in the 25-hydroxyvitamin D antibody solution has a concentration of 0.1 mg/mL to 0.6 mg/mL, specifically 0.1 mg/mL, 0.2 mg/mL, 0.3 mg/mL, 0.4 mg/mL, 0.5 mg/mL, or 0.6 mg/mL; the 25-hydroxyvitamin D antibody may be a 25-hydroxyvitamin D monoclonal antibody or a 25-hydroxyvitamin D polyclonal antibody, preferably the 25-hydroxyvitamin D monoclonal antibody; the 25-hydroxyvitamin D antibody solution may be prepared from the 25-hydroxyvitamin D antibody and a PBS buffer, and the PBS buffer is specifically 1×PBS, pH-7.4; the 25-hydroxyvitamin D antibody is purchased from Nanjing OKay Biotechnology Co., Ltd. China, product No. R484j4, with a purity greater than 95%. In an embodiment, the coupling may be conducted at a temperature of 30° C. to 40° C., specifically 37° C.; the coupling may be conducted for 1 h to 4 h, specifically 2 h; the coupling is preferably conducted under shaking. In examples of the present disclosure, the aldehyde groups on the activated glass rod can undergo condensation with the amino and carboxyl groups on the 25-hydroxyvitamin D antibody, thereby coupling the 25-hydroxyvitamin D antibody onto the glass rod.

In examples of the present disclosure, each 25-hydroxyvitamin D antibody molecule has 2 antigen binding sites, and an antibody-to-antigen molar ratio at an antibody-antigen saturation state is 1:2. Therefore, during the antibody coupling, a sufficient amount of antibody is coupled to the surface of the glass rod according to the specific detection requirements to ensure that all targets could be extracted, while a sufficient glass surface area needs to be provided for coupling antibodies. Considering a production cost of the extraction rod, the amount of antibody should be as small as possible. Inventor(s) has calculated theoretically and verified experimentally that a μg-level (<10 μg) antibody coupled to each glass rod could fully meet the detection requirements of actual biological samples (such as serum samples), and a cost of antibody in such amount could also meet the actual detection requirements.

In the present disclosure, after obtaining the antibody-coupled glass rod, the antibody-coupled glass rod is subjected to deactivation to obtain a deactivated glass rod. In an embodiment, the antibody-coupled glass rod is placed in a deactivation reagent for deactivation, for example, a treatment end of the antibody-coupled glass rod may be placed in the deactivation reagent for deactivation, and an immersion depth of the treatment end in the deactivation agent may be not less than 0.5 cm, specifically 1 cm to 1.5 cm. In an embodiment, the deactivation reagent may be an aqueous ethanolamine solution; ethanolamine in the aqueous ethanolamine solution may have a concentration of 0.25 mol/L to 0.35 mol/L, specifically 0.3 mol/L; the aqueous ethanolamine solution may have a pH value of 7 to 7.5, specifically the pH value of the aqueous ethanolamine solution may be adjusted with concentrated hydrochloric acid. In an embodiment, the deactivation may be conducted at a temperature of 20° C. to 30° C., specifically room temperature; the deactivation may be conducted for 1.5 h to 2.5 h, specifically 2 h. The deactivation could remove the remaining aldehyde groups on the surface of the glass rod.

In the present disclosure, the deactivated glass rod is subjected to covalent bond fixation to obtain the extraction rod. In an embodiment, the deactivated glass rod is placed in a reducing agent for covalent bond fixation, for example, a treatment end of the deactivated glass rod may be placed in the reducing agent for covalent bond fixation, and an immersion depth of the treatment end in the reducing agent may be not less than 0.5 cm, specifically 1 cm to 1.5 cm; the reducing agent may be prepared from sodium cyanoborohydride and a PBS buffer, and sodium cyanoborohydride in the reducing agent has a concentration of 0.15 mg/mL to 0.25 mg/mL, specifically 2 mg/mL; the PBS buffer may have a pH value of 7.0 to 7.4, specifically 7.0 in an example. In an embodiment, the covalent bond fixation may be conducted at a temperature of 3° C. to 5° C., specifically 4° C.; the covalent bond fixation may be conducted for 40 h to 60 h, specifically 48 h. An imine bond formed by the condensation may be reduced to an amine bond by the sodium cyanoborohydride, thereby stabilizing the covalent bond. In an embodiment, the extraction rod may be stored at 4° C. in a PBS buffer (with a pH value of 7.0 to 7.4) containing 0.02 wt % sodium azide.

The present disclosure further provides an extraction rod prepared by the method.

The present disclosure further provides a method for detecting 25-hydroxyvitamin D for a non-diagnostic purpose, including the following steps:
mixing a biological sample to be tested with a dissociation agent, and subjecting a resulting mixture to dissociation to obtain a dissociation liquid; where the dissociation agent includes 8-anilino-1-naphthalenesulfonic acid and perfluorooctanoic acid (PFOA);
placing the extraction rod as described in above technical solutions in the dissociation liquid, and conducting extraction to obtain an enriched extraction rod;
subjecting the enriched extraction rod to cleaning and elution in sequence to obtain a solution to be tested;
subjecting the solution to be tested to liquid chromatography-tandem mass spectrometry (LC-MS/MS) detection to obtain a chromatogram of the solution to be tested; and
obtaining a content of the 25-hydroxyvitamin D in the biological sample to be tested according to a standard curve of the 25-hydroxyvitamin D and the chromatogram of the solution to be tested.

In the present disclosure, an extraction rod based on antibody immune enrichment is prepared by combining the advantages of both the immune method and the mass spectrometry. The extraction rod is used in conjunction with LC-MS/MS to determine the 25-hydroxyvitamin D (specifically including 25-hydroxyvitamin D2 and 25-hydroxyvitamin D3), and could effectively reduce matrix interference and improve the specificity and accuracy of detection. In addition, the extraction rod has a simple pretreatment process, does not require nitrogen blowing, centrifugation and other operations, consumes little reagents, and is easy to realize automated operation. Therefore, this extraction rod is suitable for rapid processing and detection of large quantities of samples, and is particularly suitable for clinical promotion and popularization. The method of the present disclosure will be described in detail below.

In the present disclosure, a biological sample to be tested is mixed with a dissociation agent, and a resulting mixture is subjected to dissociation to obtain a dissociation liquid. In an embodiment, the biological sample to be tested may include serum, plasma, urine, or tissue fluid. In examples of the present disclosure, the serum is used as an example to verify the detection effect of solutions of the present disclosure. Without any other treatment, the serum sample to be tested may be directly mixed with the dissociation agent for dissociation. The dissociation agent includes 8-anilino-1-naphthalenesulfonic acid and PFOA. In an embodiment, the 8-anilino-1-naphthalenesulfonic acid in the dissociation agent may have a concentration of 0.1 wt % to 0.5 wt %, specifically 0.1 wt %, 0.15 wt %, 0.18 wt %, 0.2 wt %, 0.3 wt %, 0.4 wt %, or 0.5 wt %; the PFOA may have a concentration of 1% to 5%, specifically 1 wt %, 1.5 wt %, 1.81 wt %, 2 wt %, 3 wt %, 4 wt %, or 5 wt %; the dissociation agent may be prepared from the 8-anilino-1-naphthalenesulfonic acid, the PFOA, and a buffered salt solution, and the buffered salt solution may be prepared from bis(2-hydroxyethyl)amino (trihydroxymethyl) methane (BIS-TRIS), sodium chloride, N,N-dimethylformamide (DMF), dimethyl sulfoxide (DMSO), and water, where the BIS-TRIS in the buffered salt solution may have a concentration of 2 wt % to 3 wt %, specifically 2.5 wt %; the sodium chloride may have a concentration of 0.5 wt % to 1.0 wt %, specifically 0.8 wt %; the DMF may have a concentration of 8.0 wt % to 8.5 wt %, specifically 8.33 wt %; the DMSO may have a concentration of 1.4 wt % to 1.8 wt %, specifically 1.6 wt %; the dissociation agent may have a pH value of 7.0 to 7.5, and hydrochloric acid may be used to adjust the pH value of the dissociation agent. In an embodiment, taking the biological sample to be tested being the serum sample as an example, a volume ratio of the serum sample to the dissociation agent may be in a range of 1:(0.8-1.2), specifically 1:1. In an embodiment, the dissociation may be conducted at a temperature of 30° C. to 40° C., specifically 37° C.; the dissociation may be conducted for 10 min to 30 min, specifically 10 min to 15 min. The 25-hydroxyvitamin D in the biological sample to be tested, such as the serum sample, can be combined with a vitamin D binding protein or other proteins to form a bound state, and an antibody on the extraction rod cannot bind to the antigen at a bound state. The dissociation agent dissociates 25-hydroxyvitamin D from the vitamin D binding protein, changing from the bound state into a free state, which is beneficial for the antibody to capture the antigen. Therefore, the selection of a suitable dissociation agent is crucial to a detection sensitivity of the biological sample to be tested. The neutral dissociation agent is prepared by using 8-anilino-1-naphthalenesulfonic acid and PFOA, has high dissociation efficiency, and does not affect the activity of the antibody.

In the present disclosure, after obtaining the dissociation liquid, the extraction rod as described in above technical solutions is placed in the dissociation liquid, and extraction is conducted to obtain an enriched extraction rod. In an embodiment, the extraction may be conducted at a temperature of 30° C. to 40° C., specifically 37° C.; the extraction may be conducted for 5 min to 30 min, specifically 20 min.

In the present disclosure, after obtaining the enriched extraction rod, the enriched extraction rod is subjected to cleaning and elution in sequence to obtain a solution to be tested. In an embodiment, a cleaning solution for the cleaning may be water or an aqueous methanol solution, and the aqueous methanol solution has a concentration less than or equal to 10 wt %, specifically 3 wt % to 5 wt %. The cleaning is conducted to ensure sufficient washing.

In an embodiment, an eluent for the elution may be methanol, acetonitrile, or a solution of a derivating agent, specifically the solution of the derivating agent. There is a poor ionization efficiency of the 25-hydroxyvitamin D under the ESI source, which affects the detection sensitivity. By using the derivating agent solution as an eluent, new derivatives can be generated through derivation, thereby improving the ionization efficiency and further improving the sensitivity. In an embodiment, the derivating agent solution may be prepared from a derivating agent and acetonitrile; the derivating agent may include PTAD, DMEQ-TAD, or DAPTAD, specifically the PTAD, and a concentration of the derivating agent solution may be in a range of 0.1 mg/mL to 1 mg/mL, specifically 0.3 mg/mL to 0.5 mg/mL. The derivating agent can form an adduct with a vitamin D compound having a cis-triene system. In an embodiment, taking the biological sample to be tested being the serum sample as an example, a volume ratio of the serum sample to the eluent may be in a range of 1:(0.8-1.2), specifically 1:1. In an embodiment, the elution may be conducted at a temperature of 30° C. to 40° C., specifically 37° C.; the elution may be conducted for 10 min to 30 min, specifically 25 min to 30 min. In an embodiment, after the elution is completed, the extraction rod is taken out and the obtained liquid is mixed with water to obtain the solution to be tested. In an embodiment, taking the biological sample to be tested being the serum sample as an example, a volume ratio of the serum sample to the water may be in a range of 1:(0.4-0.6), specifically 1:0.5; the mixing may be performed by vortex mixing, and the vortex mixing may be conducted for 30 s. In an embodiment, the extraction rod may be regenerated after use to restore its antibody activity, and a regeneration process includes: washing the used extraction rod in methanol 2 times to remove the target analyte remained on the extraction rod, and then placing the washed extraction rod in a PBS solution containing 0.02 wt % sodium azide and storing for later use.

In the present disclosure, after obtaining the solution to be tested, the solution to be tested is subjected to LC-MS/MS detection to obtain a chromatogram of the solution to be tested. In an embodiment, conditions of liquid phase chromatography in the LC-MS/MS detection may include: a mobile phase including a mobile phase A and a mobile phase B, the mobile phase A being an aqueous ammonium acetate solution with a concentration of 2 mmol/L, and the mobile phase B being methanol; and a gradient elution program including: a volume fraction of the mobile phase B being 10% from 0 min to 1 min; increasing the volume fraction of the mobile phase B from 10% to 100% from 1 min to 3.5 min; maintaining the volume fraction of the mobile phase B at 100% from 3.5 min to 4.5 min; decreasing the volume fraction of the mobile phase B from 100% to 10% from 4.5 min to 4.6 min; and maintaining the volume fraction of the mobile phase B at 10% from 4.6 min to 5 min. In an embodiment, conditions of liquid phase chromatography in the LC-MS/MS detection may also include: a chromatographic column being specifically a Waters BEH C18 column (2.1×50 mm, 1.7 μm); a mobile phase flow rate being specifically 0.2 mL/min; a column temperature being specifically 40° C.; and an injection volume being specifically 5 μL.

In an embodiment of the present disclosure, conditions of mass spectrometry in the LC-MS/MS detection may include: an acquisition ion mode: electrospray ionization (ESI) positive ion mode; a capillary voltage: 3.6 kV; a desolvation gas temperature: 500° C.; a desolvation gas flow rate: 1,000 L/h; a cone gas flow rate: 150 L/h; a scanning mode: multiple reaction monitoring (MRM) mode (the specific MRM parameters are shown in Table 1 in Example 1 and will not be repeated here). In an embodiment, conditions of mass spectrometry in the LC-MS/MS detection may also include: a mass spectrometer being Waters TQ-Absolute.

In the present disclosure, after obtaining the chromatogram of the solution to be tested, a content of the 25-hydroxyvitamin D in the biological sample to be tested is obtained according to a standard curve of the 25-hydroxyvitamin D and the chromatogram of the solution to be tested. In an embodiment, the contents of 25-hydroxyvitamin D2 and 25-hydroxyvitamin D3 in the biological sample to be tested are obtained based on the standard curve of 25-hydroxyvitamin D2 and the standard curve of 25-hydroxyvitamin D3 and the chromatogram of the solution to be tested, respectively. There is no particular limitation on a process for obtaining the standard curve of the 25-hydroxyvitamin D, and a method well known to those skilled in the art may be used. In examples of the present application, the standard curve of the 25-hydroxyvitamin D is specifically a linear equation of the mass concentration of 25-hydroxyvitamin D and the chromatographic peak area. Specifically, the standard curve of 25-hydroxyvitamin D takes the chromatographic peak area of 25-hydroxyvitamin D as an ordinate and the mass concentration of 25-hydroxyvitamin D as an abscissa. In examples of the present disclosure, the standard solution of the 25-hydroxyvitamin D is subjected to dissociation, extraction, washing, elution, and LC-MS/MS detection according to the above solutions to obtain the chromatographic peak area of 25-hydroxyvitamin D; a standard curve is plotted based on the chromatographic peak area of 25-hydroxyvitamin D and the mass concentration of 25-hydroxyvitamin D. In an embodiment of the present disclosure, the standard solution of 25-hydroxyvitamin D may be prepared from 25-hydroxyvitamin D and a bovine serum albumin (BSA) solution, and the BSA solution may have a concentration of 4 wt %; the standard solution of 25-hydroxyvitamin D may have a concentration of 1 ng/mL, 2 ng/mL, 5 ng/ml, 10 ng/ml, 20 ng/ml, 50 ng/ml, and 100 ng/mL. According to the standard curve of 25-hydroxyvitamin D and the chromatogram of the solution to be tested, the content of 25-hydroxyvitamin D in the solution to be tested could be obtained, and then the content of 25-hydroxyvitamin D in the biological sample to be tested could be obtained. The standard curve of 25-hydroxyvitamin D2 and the standard curve of 25-hydroxyvitamin D3 in the examples can be obtained according to the above method, which will not be repeated here.

The technical solutions of the present disclosure will be clearly and completely described below in conjunction with the examples of the present disclosure. Apparently, the described examples are merely a part rather than all of the examples of the present disclosure. All other examples obtained by those skilled in the art based on the examples of the present disclosure without creative efforts shall fall within the scope of the present disclosure.

A high borosilicate glass rod used in the following examples was purchased from Yongsheng Quartz Products Store, China, and the high borosilicate glass rod has a diameter of 3 mm and a length of 60 mm.

### Example 1

A vitamin D affinity extraction rod was prepared as follows:

At a volume ratio of aqueous hydrogen peroxide solution (with a concentration of 30 wt %) to concentrated sulfuric acid (with a concentration of 95 wt % to 98 wt %) of 3:7, the aqueous hydrogen peroxide solution was added to the concentrated sulfuric acid to obtain a piranha solution. At room temperature, one end of a high borosilicate glass rod (referred to as the treatment end) was placed in the piranha solution (with an immersion depth of 1 cm) and etched for 1 h, followed by thoroughly rinsing with pure water, anhydrous ethanol, and pure water in sequence to obtain an etched glass rod.

3-Aminopropyltriethoxysilane, water, and anhydrous ethanol were mixed at a volume ratio of 5:5: 90 to obtain a silane modification solution. The etched glass rod was placed in the silane modification solution (the treatment end was immersed to a depth of 1 cm), and silanized for 24 h at room temperature, followed by thoroughly rinsing with pure water and anhydrous ethanol in sequence to obtain a silanized glass rod.

The silanized glass rod was placed in a tubular furnace, in a protective atmosphere of nitrogen, and subjected to heat treatment at 80° C. for 12 h, followed by taking out and then cooling to obtain a heat-treated glass rod.

The heat-treated glass rod was placed in a PBS solution (pH=7.0) containing 2.5 wt % glutaraldehyde (the treatment end was immersed to a depth of 1 m), and activated at room temperature for 5 hours, followed by thoroughly rinsing with pure water to obtain an activated glass rod.

The activated glass rod was placed in a PBS solution (pH=7.0) containing 0.5 mg/mL vitamin D monoclonal antibody (the treatment end was immersed to a depth of 0.5 cm) and oscillated at 37° C. for 2 h. An obtained glass rod was placed in an ethanolamine solution with a concentration of 0.3 mol/L and a pH value of 7.5 (the pH value was adjusted with concentrated hydrochloric acid, and the treatment end was immersed to a depth of 1 cm) and placed at room temperature for 2 h to deactivate the remaining aldehyde groups. A resulting glass rod was placed in a PBS solution (pH=7.0) containing 0.2 mg/mL sodium cyanoborohydride (the treatment end was immersed to a depth of 1 cm) and placed at 4° C. for 48 h to reduce the imine to amine, thereby obtaining a vitamin D antibody immunoaffinity extraction rod (referred to as the extraction rod). The extraction rod was placed in a PBS solution (pH=7.0) containing 0.02 wt % sodium azide and stored at 4° C.

### Example 2

The extraction rod in Example 1 was used to extract and detect serum 25-hydroxyvitamin D, which was performed as follows:

I. The sample pretreatment steps were as follows:

(1) 100 μL of 25-hydroxyvitamin D standard solution or serum sample was added into a 1.5 mL centrifuge tube, and then 100 μL of dissociation agent was added thereto, and a resulting mixture was mixed by vortex, and then incubated under shaking at 37° C. for 10 min; where the dissociation agent was a neutral dissociation agent prepared from 8-anilino-1-naphthalenesulfonic acid, PFOA, and buffer solution (a pH value thereof was adjusted to 7 using hydrochloric acid), and the concentration of 8-anilino-1-naphthalenesulfonic acid in the dissociation agent was 0.18 wt %, and the concentration of PFOA was 1.81 wt %; the buffer solution was composed of bis(2-hydroxyethyl)amino (trihydroxymethyl) methane (BIS-TRIS), sodium chloride, DMF, DMSO, and water, and the contents of each component were 2.5 wt % BIS-TRIS, 0.8 wt % sodium chloride, 8.33 wt % DMF, 1.6 wt % DMSO, and water as a balance.

(2) The extraction rod was placed in a centrifuge tube and incubated under shaking at 37° C. for 20 min, followed by taking out and then washing with a 5 wt % aqueous methanol solution.

(3) The washed extraction rod was placed in an inner tube of an injection bottle containing 100 μL of 0.5 mg/mL solution of 4-phenyl-1,2,4-triazolidine-3,5-dione (PTAD) in acetonitrile, and eluted and subjected derivative reaction at 37° C. for 30 min.

(4) 50 μL of pure water was added into the inner tube of the injection bottle and mixed by vortex for LC-MS/MS detection.

II. Detection conditions of chromatography and mass spectrometry were as follows:

1. Conditions of chromatography:

Chromatographic column: Waters BEH C18 column (2.1×50 mm, 1.7 μm).

Mobile phase A: 2 mM aqueous ammonium acetate solution; mobile phase B: pure methanol.

A gradient elution program: at 0 min, a volume fraction of mobile phase B was 10%; at 1 min, the volume fraction of mobile phase B remained at 10%; at 3.5 min, the volume fraction of mobile phase B was 100%; at 4.5 min, the volume fraction of mobile phase B remained at 100%; at 4.6 min, the volume fraction of mobile phase B was 10%; at 5 min, the volume fraction of mobile phase B remained at 10%.

Mobile phase flow rate: 0.2 mL/min; column temperature: 40° C.; injection volume: 5 μL.

2. Conditions of mass spectrometry:

Mass spectrometer and acquisition ion mode: Waters TQ-Absolute, ESI positive ion mode.

A capillary voltage: 3.6 kV; a desolvation gas temperature: 500° C.; a desolvation gas flow rate: 1,000 L/h; a cone gas flow rate: 150 L/h.

A scanning mode: MRM mode, and mass spectrometry parameters of each compound were shown in Table 1.

TABLE 1
Mass spectrometry MRM parameters of 25-hydroxyvitamin D

| | | | | Collision energy |
|---|---|---|---|---|
| Compound name | Parent ion | Daughter ion | Cone voltage (V) | (V) |
| 25-hydroxyvitamin D3 | 558.4 | 280.1 | 20 | 28 |
| 25-hydroxyvitamin D3 | 558.4 | 298.2 | 20 | 10 |
| 25-hydroxyvitamin D2 | 570.4 | 280.1 | 20 | 28 |
| 25-hydroxyvitamin D2 | 570.4 | 298.0 | 20 | 14 |

### Example 3

Serum samples and 4 wt % BSA solution samples were separately added with 25-hydroxyvitamin D standards to make their concentrations 5 ng/ml, 10 ng/ml, 20 ng/ml, and 50 ng/mL, respectively, and then treated according to the sample pretreatment steps in Example 2, followed by LC-MS/MS detection under conditions of chromatography and mass spectrometry in Example 2.

Table 2 shows peak areas of 25-hydroxyvitamin D extracted from different samples with the extraction rod when the dissociation agent was added. The results show that the extraction rod could extract 25-hydroxyvitamin D in both serum samples and 4 wt % BSA solution samples. Moreover, at each addition concentration, the response of the serum sample after background deduction is close to that of the 4 wt % BSA solution sample. This indicates that the dissociation agent could fully dissociate 25-hydroxyvitamin D in the serum sample.

TABLE 2
Peak areas of 25-hydroxyvitamin D extracted from different samples

| | | | Sample type | |
|---|---|---|---|---|
| Target compound | Addition concentration | | 4 wt % BSA solution | Serum |
| 25-hydroxyvitamin D2 | Not added | | 42.6 | 443.1 |
| | 5 | ng/mL | 3321.3 | 3456.7 |
| | 10 | ng/mL | 6524.6 | 7312.5 |
| | 20 | ng/mL | 10458.5 | 11637.8 |
| | 50 | ng/mL | 24105.8 | 27082.8 |
| 25-hydroxyvitamin D3 | Not added | | 62.7 | 78416.8 |
| | 5 | ng/mL | 6644.9 | 83335.6 |
| | 10 | ng/mL | 13781.7 | 93519 |
| | 20 | ng/mL | 20627.1 | 98885.5 |
| | 50 | ng/mL | 50290.5 | 136097.9 |

### Example 4

25-hydroxyvitamin D2 standard solution and 25-hydroxyvitamin D3 standard solution were prepared with the BSA solution having a concentration of 4 wt %, and the concentrations were 1 ppb, 2 ppb, 5 ppb, 10 ppb, 20 ppb, 50 ppb, and 100 ppb, respectively. The samples were treated according to the sample pretreatment steps in Example 2, and LC-MS/MS detection was conducted under conditions of chromatography and mass spectrometry in Example 2 to obtain the retention time and linear range of 25-hydroxyvitamin D2 and 25-hydroxyvitamin D3.

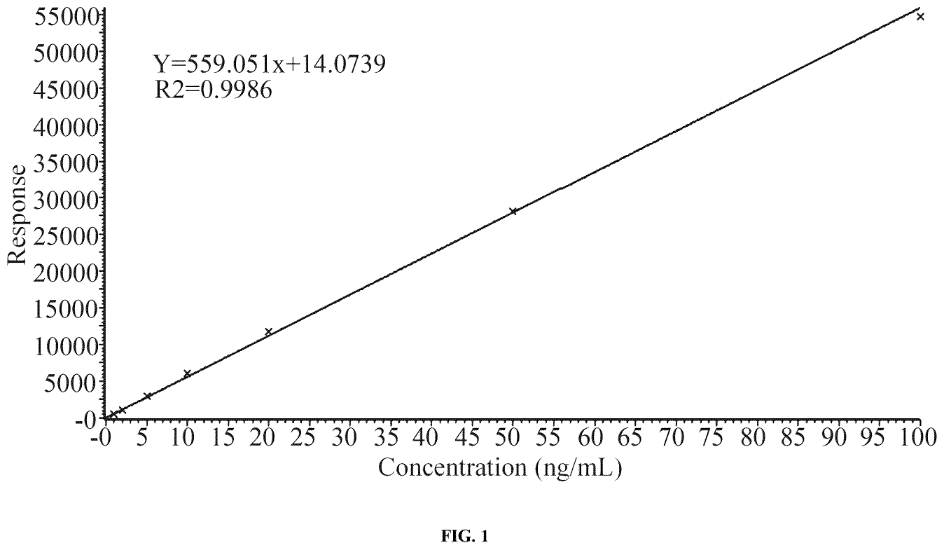

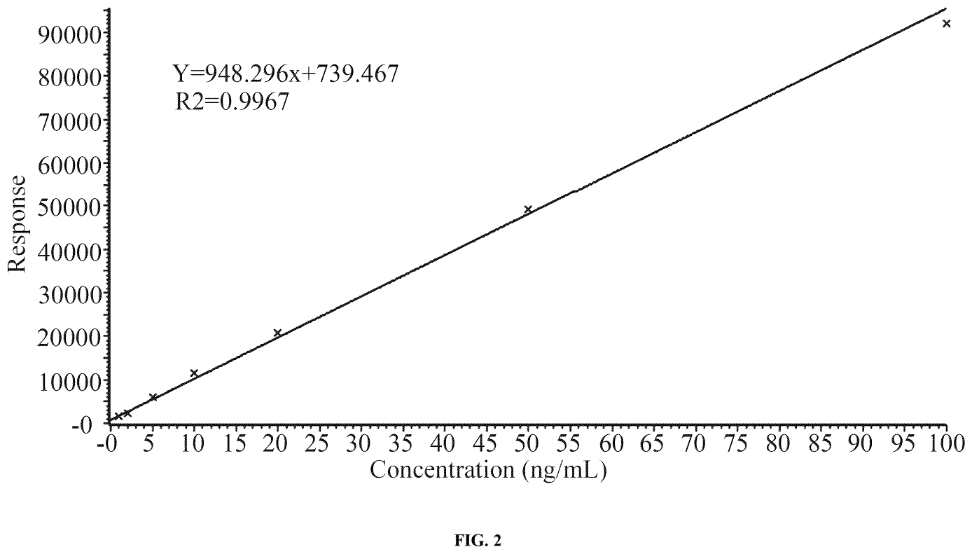

Table 3 shows the retention time and linear range of 25-hydroxyvitamin D2 and 25-hydroxyvitamin D3, FIG. 1 shows a standard curve of 25-hydroxyvitamin D2, and FIG. 2 shows a standard curve of 25-hydroxyvitamin D3. The results show that the linear correlation coefficient R² of 25-hydroxyvitamin D2 and 25-hydroxyvitamin D3 satisfies R²>0.99, indicating that it is acceptable within the linear range and meets the actual experimental requirements.

TABLE 3
Retention time and linear range of 25-hydroxyvitamin D2 and 25-hydroxyvitamin D3

| | | | | Correlation |
|---|---|---|---|---|
| | Retention time | Linear range | | coefficient |
| Target compound | (min) | (ng/mL) | Linear equation | (R²) |
| 25-hydroxyvitamin | 2.53 | 1-100 | Y = 559.051x + 14.0739 | 0.9986 |
| D2 | | | | |
| 25-hydroxyvitamin | 2.53 | 1-100 | Y = 948.296x + 739.467 | 0.9967 |
| D3 | | | | |

### Example 5

Low-, medium-, and high-concentration 25-hydroxyvitamin D standards were separately added into the serum samples to obtain experimental group samples, which were then treated according to the sample pretreatment steps in Example 2, followed by LC-MS/MS detection under conditions of chromatography and mass spectrometry in Example 2. The basic sample concentration in the serum samples and the sample concentration of each experimental group were calculated according to the standard curve. The recovery rate=(detected concentration−basic sample concentration)/theoretical addition concentration×100%.

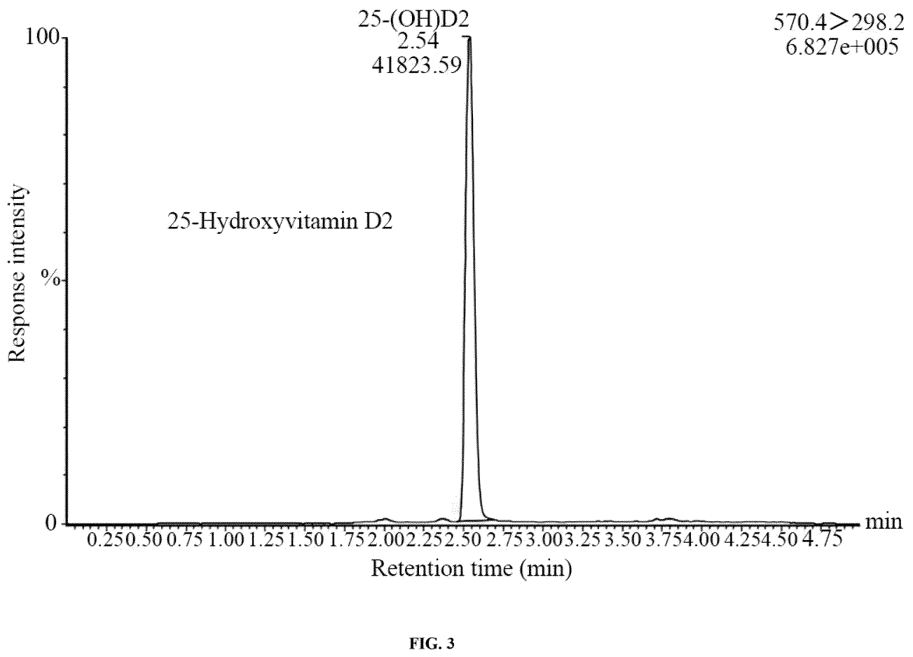

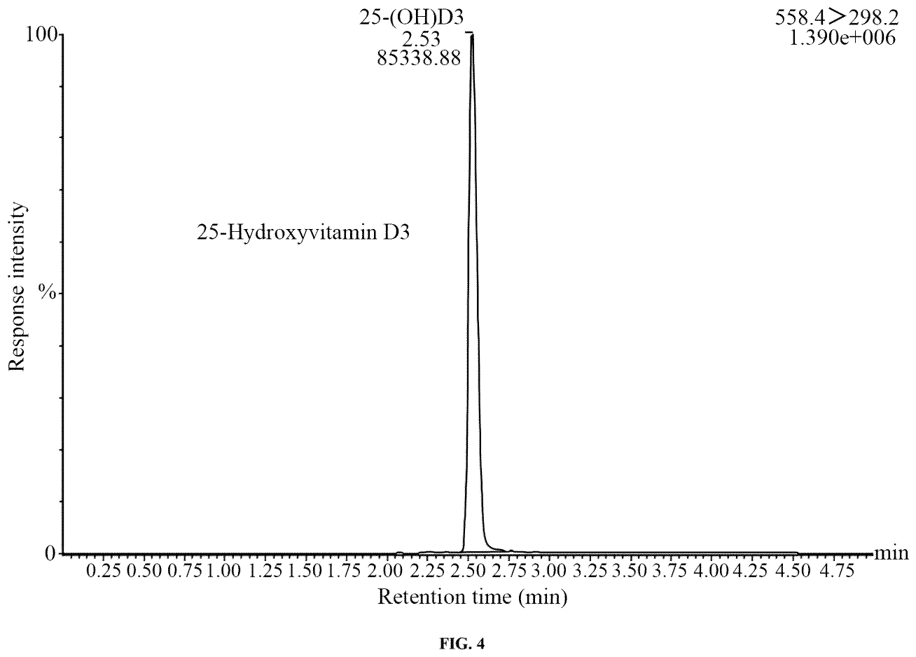

FIG. 3 shows a chromatogram obtained by LC-MS/MS detection of the 25-hydroxyvitamin D2 in a spiked serum sample after extraction with the extraction rod; and FIG. 4 shows a chromatogram obtained by LC-MS/MS detection of the 25-hydroxyvitamin D3 in a spiked serum sample after extraction with the extraction rod, where a spike concentration was 50 ng/mL. The detection results of the spiked serum samples are shown in Table 4. The results show that the spiked recovery rates of low-, medium- and high-concentration 25-hydroxyvitamin D in serum samples all faill within a range of 85% to 115%, meeting the actual experimental requirements.

TABLE 4
Spiked recovery rate of 25-hydroxyvitamin D

| | Basic | Low-concentration | | Medium-concentration | | High-concentration | |
|---|---|---|---|---|---|---|---|
| | sample | spike (5 ng/mL) | | spike (20 ng/mL) | | spike (50 ng/mL) | |
| Target | concentration | Detected | Recovery | Detected | Recovery | Detected | Recovery |
| compound | (ng/mL) | concentration | rate | concentration | rate | concentration | rate |
| 25-hydroxyvitamin | 0.9 | 6.2 | 106% | 20.2 | 96.5% | 50.5 | 99.2% |
| D2 | | | | | | | |
| 25-hydroxyvitamin | 83.3 | 88.0 | 94% | 105.3 | 110% | 140.2 | 113.8% |
| D3 | | | | | | | |

### Comparative Example 1

The dissociation effects of the 4 dissociation agents were compared as follows:
Dissociation agent 1: commercially available dissociation agent (purchased from Nanjing OKay Biotechnology Co., Ltd. China, product number D10a1);
Dissociation agent 2: prepared from PFOA and an ethylene glycol-containing PBS solution, where the concentration of ethylene glycol in the ethylene glycol-containing PBS solution was 10 wt %, and the concentration of PFOA in the dissociation agent 2 was 2 wt %;
Dissociation agent 3: perfluorohexanoic acid was mixed with a 5 wt % aqueous methanol solution, and then the pH value of the resulting mixture was adjusted to 7.5 using sodium hydroxide, and the concentration of perfluorohexanoic acid in the dissociation agent 3 was 0.75 wt %;
Dissociation agent 4: the dissociation agent used in Example 2.

The same amount of 25-hydroxyvitamin D standard was separately added into 4 serum samples, and the samples were then treated according to the sample pretreatment steps in Example 2, where the dissociation agents used were the dissociation agents 1, 2, 3, and 4 above, respectively, and LC-MS/MS detection was conducted under conditions of chromatography and mass spectrometry in Example 2.

Table 5 shows peak areas of 25-hydroxyvitamin D extracted from serum samples with the extraction rod when different dissociation agents are added. The results show that for the same sample, the highest response of 25-hydroxyvitamin D is detected after dissociation with dissociation agent 4, indicating that dissociation agent 4 has the best dissociation ability, while other dissociation agents have poor dissociation efficiency and result in incomplete dissociation.

TABLE 5
Peak areas of 25-hydroxyvitamin D extracted from serum
samples when different dissociation agents are added

| | Dissociation | Dissociation | Dissociation | Dissociation |
|---|---|---|---|---|
| Target compound | agent 1 | agent 2 | agent 3 | agent 4 |
| 25-hydroxyvitamin D2 | 10588 | 1356 | 5955 | 19308 |
| 25-hydroxyvitamin D3 | 47404 | 3249 | 11941 | 63983 |

### Comparative Example 2

The serum samples and the BSA solution samples with a concentration of 4 wt % were separately added with the same amount of 25-hydroxyvitamin D. The samples were treated according to the sample pretreatment steps in Example 2 expect that the dissociation agent was not added, and LC-MS/MS detection was conducted under conditions of chromatography and mass spectrometry in Example 2.

Table 6 shows peak areas of 25-hydroxyvitamin D extracted from different samples without adding a dissociation agent. The results show that the extraction rod could extract 25-hydroxyvitamin D in a BSA solution sample with a concentration of 4 wt %, but 25-hydroxyvitamin D is not extracted from a serum sample. The reason lies in that after 25-hydroxyvitamin D is added to the serum sample, it might bind to proteins such as vitamin D binding protein in the serum to form a bound state, and could not be extracted by the antibody on the extraction rod.

TABLE 6
Peak areas of 25-hydroxyvitamin D extracted from different
samples without adding dissociation agent

Sample type

| Target compound | 4 wt % BSA solution | Serum |
|---|---|---|
| 25-hydroxyvitamin D2 | 4357 | 54 |
| 25-hydroxyvitamin D3 | 13892 | 260 |

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. A method for preparing an extraction rod, comprising:
subjecting a glass rod to etching, silanization, a heat treatment, and activation in sequence to obtain an activated glass rod;
placing the activated glass rod in a 25-hydroxyvitamin D antibody solution, and conducting coupling to obtain an antibody-coupled glass rod; and
subjecting the antibody-coupled glass rod to deactivation and covalent bond fixation in sequence to obtain the extraction rod.

2. The method as claimed in claim 1, wherein a 25-hydroxyvitamin D antibody in the 25-hydroxyvitamin D antibody solution has a concentration of 0.1 mg/mL to 0.6 mg/mL; and the 25-hydroxyvitamin D antibody is selected from the group consisting of a 25-hydroxyvitamin D monoclonal antibody and a 25-hydroxyvitamin D polyclonal antibody.

3. The method as claimed in claim 1, wherein the coupling is conducted at a temperature of 30° C. to 40° C. for 1 hour to 4 hours.

4. The method as claimed in claim 2, wherein the coupling is conducted at a temperature of 30° C. to 40° C. for 1 hour to 4 hours.

5. An extraction rod prepared by the method as claimed in claim 1.

6. A method for detecting 25-hydroxyvitamin D for a non-diagnostic purpose, comprising:
mixing a biological sample to be tested with a dissociation agent, and subjecting a resulting mixture to dissociation to obtain a dissociation liquid, wherein the dissociation agent comprises 8-anilino-1-naphthalenesulfonic acid and perfluorooctanoic acid (PFOA);
placing the extraction rod as claimed in claim 5 in the dissociation liquid, and conducting extraction to obtain an enriched extraction rod;
subjecting the enriched extraction rod to cleaning and elution in sequence to obtain a solution to be tested;
subjecting the solution to be tested to liquid chromatography-tandem mass spectrometry (LC-MS/MS) detection to obtain a chromatogram of the solution to be tested; and
obtaining a content of the 25-hydroxyvitamin D in the biological sample to be tested according to a standard curve of the 25-hydroxyvitamin D and the chromatogram of the solution to be tested.

7. The method as claimed in claim 6, wherein the 25-hydroxyvitamin D comprises 25-hydroxyvitamin D2 and 25-hydroxyvitamin D3.

8. The method as claimed in claim 6, wherein in the dissociation agent, the 8-anilino-1-naphthalenesulfonic acid has a concentration of 0.1 wt % to 0.5 wt % and the PFOA has a concentration of 1 wt % to 5 wt %;
the dissociation agent has a pH value of 7.0 to 7.5; and
the dissociation is conducted at a temperature of 30° C. to 40° C. for 10 minutes to 30 minutes.

9. The method as claimed in claim 6, wherein the extraction is conducted at a temperature of 30° C. to 40° C. for 5 minutes to 30 minutes;
an eluent for the elution comprises one selected from the group consisting of methanol, acetonitrile, and a solution of a derivating agent, and the derivating agent comprises one selected from the group consisting of 4-phenyl-1,2,4-triazoline-3,5-dione, 4-(2-(6,7-dimethoxy-4-methyl-3-oxo-3,4-dihydro-quinoxalin-2-yl)-ethyl)-(1,2,4) triazoline-3,5-di one, and 4-(4′-dimethylaminophenyl)-1,2,4-triazoline-3,5-dione; and the elution is conducted at a temperature of 30° C. to 40° C. for 10 minutes to 30 minutes.

10. The method as claimed in claim 7, wherein the extraction is conducted at a temperature of 30° C. to 40° C. for 5 minutes to 30 minutes;
an eluent for the elution comprises one selected from the group consisting of methanol, acetonitrile, and a solution of a derivating agent, and the derivating agent comprises one selected from the group consisting of 4-phenyl-1,2,4-triazoline-3,5-dione, 4-(2-(6,7-dimethoxy-4-methyl-3-oxo-3,4-dihydro-quinoxalin-2-yl)-ethyl)-(1,2,4) triazoline-3,5-di one, and 4-(4′-dimethylaminophenyl)-1,2,4-triazoline-3,5-dione; and the elution is conducted at a temperature of 30° C. to 40° C. for 10 minutes to 30 minutes.

11. The method as claimed in claim 8, wherein the extraction is conducted at a temperature of 30° C. to 40° C. for 5 minutes to 30 minutes;
an eluent for the elution comprises one selected from the group consisting of methanol, acetonitrile, and a solution of a derivating agent, and the derivating agent comprises one selected from the group consisting of 4-phenyl-1,2,4-triazoline-3,5-dione, 4-(2-(6,7-dimethoxy-4-methyl-3-oxo-3,4-dihydro-quinoxalin-2-yl)-ethyl)-(1,2,4) triazoline-3,5-di one, and 4-(4′-dimethylaminophenyl)-1,2,4-triazoline-3,5-dione; and the elution is conducted at a temperature of 30° C. to 40° C. for 10 minutes to 30 minutes.

12. The method as claimed in claim 6, wherein conditions of liquid phase chromatography in the LC-MS/MS detection comprise:
a mobile phase comprising a mobile phase A and a mobile phase B, wherein the mobile phase A is an aqueous ammonium acetate solution with a concentration of 2 mmol/L, and the mobile phase B is a solution of ammonium acetate in methanol, with a concentration of 2 mmol/L; and
a gradient elution program comprising: a volume fraction of the mobile phase B being 10% from 0 minute to 1 minute; increasing the volume fraction of the mobile phase B from 10% to 100% from 1 minute to 3.5 minutes; maintaining the volume fraction of the mobile phase B at 100% from 3.5 minutes to 4.5 minutes; decreasing the volume fraction of the mobile phase B from 100% to 10% from 4.5 minutes to 4.6 minutes; and maintaining the volume fraction of the mobile phase B at 10% from 4.6 minutes to 5 minutes.

13. The method as claimed in claim 7, wherein conditions of liquid phase chromatography in the LC-MS/MS detection comprise:
a mobile phase comprising a mobile phase A and a mobile phase B, wherein the mobile phase A is an aqueous ammonium acetate solution with a concentration of 2 mmol/L, and the mobile phase B is a solution of ammonium acetate in methanol, with a concentration of 2 mmol/L; and
a gradient elution program comprising: a volume fraction of the mobile phase B being 10% from 0 minute to 1 minute; increasing the volume fraction of the mobile phase B from 10% to 100% from 1 minute to 3.5 minutes; maintaining the volume fraction of the mobile phase B at 100% from 3.5 minutes to 4.5 minutes; decreasing the volume fraction of the mobile phase B from 100% to 10% from 4.5 minutes to 4.6 minutes; and maintaining the volume fraction of the mobile phase B at 10% from 4.6 minutes to 5 minutes.

14. The method as claimed in claim 8, wherein conditions of liquid phase chromatography in the LC-MS/MS detection comprise:
a mobile phase comprising a mobile phase A and a mobile phase B, wherein the mobile phase A is an aqueous ammonium acetate solution with a concentration of 2 mmol/L, and the mobile phase B is a solution of ammonium acetate in methanol, with a concentration of 2 mmol/L; and
a gradient elution program comprising: a volume fraction of the mobile phase B being 10% from 0 minute to 1 minute; increasing the volume fraction of the mobile phase B from 10% to 100% from 1 minute to 3.5 minutes; maintaining the volume fraction of the mobile phase B at 100% from 3.5 minutes to 4.5 minutes; decreasing the volume fraction of the mobile phase B from 100% to 10% from 4.5 minutes to 4.6 minutes; and maintaining the volume fraction of the mobile phase B at 10% from 4.6 minutes to 5 minutes.

15. The method as claimed in claim 12, wherein conditions of mass spectrometry in the LC-MS/MS detection comprises:
an acquisition ion mode: electrospray ionization (ESI) positive ion mode;
a capillary voltage: 3.6 kV;
a desolvation gas temperature: 500° C.;
a desolvation gas flow rate: 1,000 L/h;
a cone gas flow rate: 150 L/h; and
a scanning mode: a multiple reaction monitoring mode.
